(19) United States
(12) Patent Application Publication
(10) Pub. No.: US 2025/0314757 A1
(43) Pub. Date: Oct. 9, 2025

(54) DISTRIBUTED SENSING WITH ULTRA-WIDEBAND RADIOS

(71) Applicant: Robert Bosch GmbH, Stuttgart (DE)

(72) Inventors: Ruofeng LIU, San Jose, CA (US); Vivek JAIN, Sunnyvale, CA (US); Christoph LANG, Sunnyvale, CA (US); Martin COORS, Sunnyvale, CA (US); Sushanta Mohan RAKSHIT, Milpitas, CA (US); Yunze ZENG, San Jose, CA (US)

(21) Appl. No.: 18/625,797

(22) Filed: Apr. 3, 2024

(51) Int. Cl. G01S 13/00; A61B 5/05; A61B 5/113; G01S 13/02; G01S 13/58

(52) U.S. Cl. CPC G01S 13/003; A61B 5/05; A61B 5/113; G01S 13/0209; G01S 13/58

(57) ABSTRACT

A distributed ultra-wide band (UWB) radar system and methods for operating the same are disclosed. The distribute radar system uses a plurality of separate UWB radios or nodes that do not use a centralized clock or source of time. However, the distributed UWB system operates to provide radar-like functionality and fine-grain sensing capabilities through synchronization based on line-of-sight (LOS) signal processing and noise estimations. Synchronized channel impulse responses (CIRs) can be processed for general object detection and tracking, gesture recognition, or even micro-motions such as monitoring vitals

### TECHNICAL FIELD

The present disclosure relates to a distributed ultra-wideband (UWB) detection technology. More specifically, an architecture that employs a unique synchronization strategy for synchronizing and aligning channel impulse responses (CIRs) communicated between separate or distributed UWB radios to provide radar-like functionality and fine-grain sensing despite not having a centralized timing source.

### BACKGROUND

Conventionally, object detection and tracking are achieved with monostatic systems such as traditional radar systems. These systems have a centralized clock/timing source. Such restraints generally result in a centralized or localized system.

### SUMMARY

A detection method comprising transmitting a UWB signal from a first (designated) transmitter mode, receiving a UWB signal at a second separate receiving node, extracting one or more (a plurality) of channel impulse responses (CIRs) from the UWB signals, synchronizing the plurality of CIRs to provide a sequence of synchronized CIRs, and processing the synchronized CIRs to detect an object or activity (e.g., movement) thereof is provided. Synchronization may comprise upsampling each CIR, aligning the plurality of CIRs, estimating a phase noise, and eliminating the phase noise from each received CIR. The phase noise may be based on the phase of a line-of-sight path signal. For example, the phase of the received line-of-sight signal may be compared to the phase of the transmitted signal.

A method of synchronizing a plurality of ultra-wideband (UWB) nodes for radar-like functionality is also provided. The method comprises upsampling each channel impulse response (CIR) of a plurality of CIRs from the UWB signal, employing a lead edge detection algorithm, aligning the plurality of CIRs based on a leading edge, determining a phase of a line-of-sight path signal for each CIR, eliminating phase error by subtracting the phase of the line-of-sight signal from each subsequent path signal.

A distributed ultra-wideband (UWB) radar system is provided. The system comprises a transmitting node and a receiving node. The transmitting node is operable to transmit a UWB signal. The receiving node operable to receive the UWB signal, extract a plurality of channel impulse responses (CIRs) from the UWB signal, synchronize the plurality of CIRs to provide a sequence of synchronized CIRs, process the synchronized CIRs to detect an object or movement, and responsive to detecting an object or movement, actuating an alert corresponding to a prediction or performing a task based on the object or movement. Synchronization may include upsampling each CIR, aligning the plurality of CIRs, estimating a phase error based on a phase of a line-of-sight path, and eliminating the phase error.

### BRIEF DESCRIPTION OF THE DRAWINGS

FIG. 1A is a schematic view of an embodiment of a monostatic architecture.

FIG. 1B is a schematic view of an embodiment of a bistatic architecture.

FIG. 1C is a schematic view of an embodiment of a multi-static environment.

FIG. 2A is a schematic view of an embodiment of a monostatic system.

FIG. 2B is a schematic view of an embodiment of a bistatic system.

FIG. 3A is perspective view of an experimental embodiment of a distributed system.

FIG. 3B is a graph illustrating CIR taps extracted from a UWB signal received by the distributed system of FIG. 3A.

FIG. 3C is a phase diagram illustrating CIR taps extracted from the UWB signal received by the distributed system of FIG. 3A.

FIG. 4A is a schematic view of an embodiment of a bi-static system having line-of-sight (LOS) and non-line of sight paths (NLOS) of a UWB signal.

FIG. 4B is a time diagram of the received CIR taps from the different paths of the embodiment for FIG. 4A.

FIG. 5 is schematic illustration of an embodiment of a multi-static system.

FIG. 6 is a flowchart illustrating operation of an embodiment of a distributed UWB detection system.

FIGS. 7A-C illustrates phase pattern analysis for breathing using DW1000 UWB radios or nodes.

FIG. 8 illustrates a micro-doppler plot for a person walking back and forth.

FIG. 9 is a flow chart illustrating a method of detection.

### DETAILED DESCRIPTION

Embodiments of the present disclosure are described herein. It is to be understood, however, that the disclosed embodiments are merely examples and other embodiments can take various and alternative forms. The figures are not necessarily to scale. Some features could be exaggerated or minimized to show details of particular components. Therefore, specific structural and functional details disclosed herein are not to be interpreted as limiting, but merely as a representative basis for teaching one skilled in the art to variously employ the embodiments of the present invention. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

This invention is not limited to the specific embodiments and methods described below, as specific components and/or conditions may vary. Furthermore, the terminology used herein is used only for the purpose of describing particular embodiments of the present invention and is not intended to be limiting in any way.

The term “substantially,” “generally,” or “about” may be used herein to describe disclosed or claimed embodiments. The terms “substantially,” “generally,” or “about” may modify a value or relative characteristic disclosed or claimed in the present disclosure to signify within manufacturing tolerances and/or within +0%, 0.1%, 0.5%, 1%, 2%, 3%, 4%, 5% or 10% of the value or relative characteristic.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

Wireless communication is overwhelmingly prevalent in modern society. However, new technologies and new uses for old technologies are still being discovered to provide greater functionality and efficiency associated with wireless communication systems. For example, wireless protocols may be used to provide traditional data communication as well as simultaneously serving as a sensing or radar-like technology. Among these is UWB radio communication. UWB technology generally has lower energy requirements but offers high bandwidth communication. Because of the low energy requirement and high communication bandwidths UWB systems are of particular interest to the automotive industry such as providing additional functionality and autonomizing automobiles. UWB communication operates through pulse-based encoding.

In UWB systems, a sequence of pulses may be transmitted to produce complex channel impulse responses (CIRs) that may be extracted from received UWB signals. The CIRs may be further processed to provide further functionality (e.g., data communication and radar-like sensing capabilities simultaneously). Given the high bandwidth of UWB and the complex nature of CIRs, rich or detailed data regarding the signaling environment may be obtained. For example, CIRs provide information about the various propagation paths, which can be useful in assessing an environment as they, for instance, reflect off the environment or objects such as people or automobiles. U.S. application Ser. No. 16/368,994 filed Mar. 29, 2019; U.S. Pat. No. 11,402,485, which issued from U.S. application Ser. No. 16/398,571 filed on Apr. 30, 2019; and U.S. Pat. No. 11,277,166, which issued from U.S. application Ser. No. 16/913,271 filed on Jun. 26, 2020 each relate to ultra-wideband sensing systems; the disclosures of which are hereby incorporated by reference in their entirety.

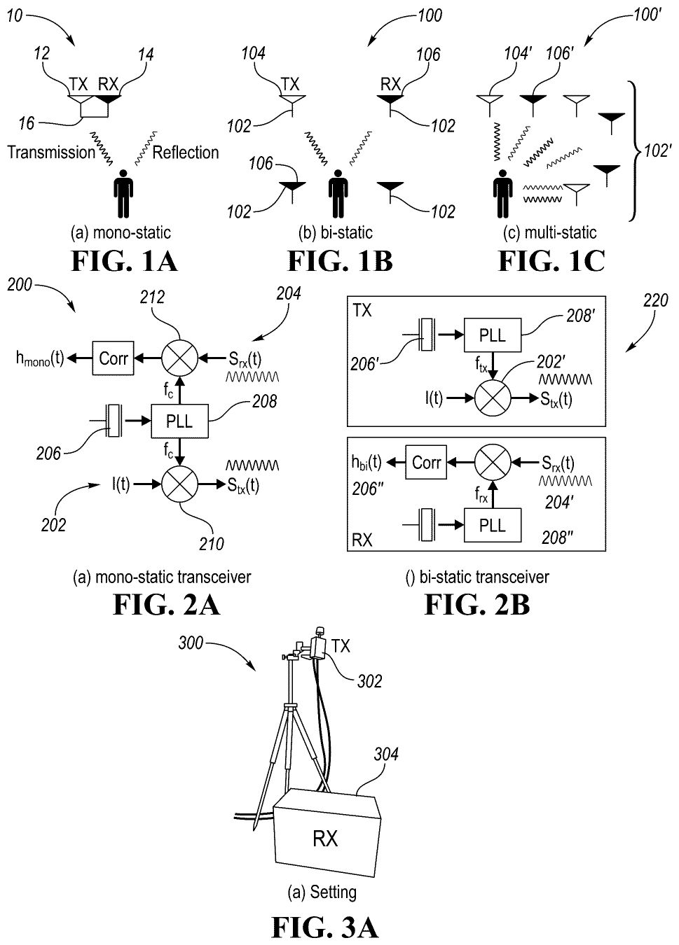

Unlike conventional monostatic sensing technologies as shown in FIG. 1A, spatially distributed UWB architectures 100, 100′ have a plurality of radio transceiver nodes 102 (e.g., at least two nodes, at least three nodes, or at least four nodes) that are spatially and physically separate or independent, as shown in FIGS. 1B-C. Conventional monostatic technologies are spatially and physically linked because they rely on a centralized clock/timing source for synchronization. For example, the transmitter 12 and receiver 14 of a monostatic transceiver are commonly in a single device. In other words, the transmitter 12 and receiver 14 of a monostatic transceiver are collocated and the transmitter(s) 104 and receiver(s) 106 of a bistatic or multi-static transceivers are not collocated. For example, monostatic architecture 10, shown FIG. 1A, has a transmitter 12, a receiver 14, and a link 16 therebetween, and bistatic/multi-static architectures 100/100′ shown in FIGS. 1B-C have no link.

Distributed UWB architectures 100, 100′ such as the bistatic (see FIG. 1B) and the multi-static (see FIG. 1C) architectures 100, 100′ do not share a centralized clock/timing source and/or physical link and thus do not have the same localization restraints. Instead, the bistatic/multi-static architectures 100, 100′ include separate independent clock/timing sources for the transmitter(s) 104, 104′ and receiver(s) 106, 106′. For instance, in various embodiments, a plurality of separate and independent radio nodes 102, 102′, each capable of transmitting and/or receiving a UWB signal, are distributed throughout the surveillance area, which generally increases the sensing area and is more robust compared with monostatic radars. The multi-static systems can also be scaled up much easier.

In various embodiments, a bistatic architecture 100, as shown in FIG. 1B, includes a transmitter node 104 that is spatially and physical separate from one or more receiver nodes 106. In a refinement, a plurality of receiver nodes 106 may be used to enhance robustness such as against specular reflection. In some embodiments, a multi-static architecture 100′, as shown in FIG. 1C, includes a plurality of transmitter nodes 104′ and a plurality of receiver nodes 106′. In the bi-static and/or multi-static system 100, 100′, a data fusion algorithm such a Kalman filter, Bayesian decision network, Dempster-Shafer framework, convolutional neural networks (CNN), and/or Gaussian processes may be used to process the various CIR streams as an aggregate. For example, a Kalman filter may be used for tracking and/or a CNN may be used for activity recognition. In some embodiments, the radio nodes 102, 102′ may operate as a transmitter node 104, 104′ and/or a receiver node 106, 106′ by both transmitting a UWB signal and receiving UWB signals. However, separate and independent clock/timing sources cause random sampling offset and random and/or uncorrelated phase noise between the separate and independent transmitter(s) 104, 104′ and receiver nodes 106, 106′ which can be problematic and difficult to overcome. This distinction is further illustrated in FIGS. 2A-B.

Referring to FIGS. 2A-B, monostatic transceiver 200 and bistatic transceiver 220 are shown. Monostatic transceiver 200 includes a transmitter 202 and a receiver 204 that are both in communication with a shared timing source such as a local oscillator, a crystal oscillator 206, and/or a phased-locked loop (PLL) 208. Bistatic transceiver 220 includes a transmitter 202′ and a receiver 204′ but transmitter 202′ is in communication with a first timing source (e.g., local oscillator, crystal oscillator 206′, and/or PLL 208′) and receiver 204′ is in communication with a second separate and distinct timing source (e.g., local oscillator, crystal oscillator 206″, and/or PLL 208″).

The monostatic transmitter 202 may produce a raw (e.g., baseband impulse) signal I(t) which may be upconverted by mixer 210 to an output (e.g., passband) signal Sₜₓ(t) having a carrier frequency fc. The carrier signal may be generated by PLL 208 using crystal oscillator 206. The output signal Sₜₓ(t) is emitted/transmitted from an antenna of the transmitter 202 (e.g., step 902), which may be characterized by formula (1):

$${S}_{tx} ( t ) = I ( t ) {e}^{j 2 π {f}_{c} t + {θ}_{0}} . \qquad ( 1 )$$

In various embodiments, t is time, j is \sqrt{−1}, and θ₀ is the initial phase. After transmission the output signal Sₜₓ may be reflected, for example, off of a single target before being received by the receiver 204 (e.g., step 904). The received signal may be characterized by formula (2):

$${S}_{rx} ( t ) = I ( t - {t}_{TOF} ) {e}^{j 2 π {f}_{c} ( t - {t}_{ToF} ) + {θ}_{0}} . \qquad ( 2 )$$

In one or more embodiments, t_{TOF} is the time of flight (i.e., delay), which may be represented by formula 3:

$${t}_{TOF} = \frac{2 d}{c} . \qquad ( 3 )$$

In various embodiments, d is the distance between the target and the transceiver and c is the speed of light. The distance of the target is represented by the time of flight as shown in formula (3) above which is determined from the amplitude spectrum, or by the phase, which is represented by formula (4):

$$\frac{4 π d}{λ} . \qquad ( 4 )$$

In various embodiments, A is the wavelength. Upon receiving the reflected signal, it is down converted back to a raw (baseband impulse) signal by mixer 212. The transmitter and receiver are collocated such that the raw (baseband impulse) signal is the same as the original raw signal (i.e., same frequency and phase). The CIR may be extracted (e.g., step 906) by cross correlating the original raw signal I(t) with the received baseband signal providing details about the environment. The CIR is characterized by formula (5):

$${h}_{mono} ( t ) = δ ( t - {t}_{ToF} ) {e}^{j 2 π {f}_{c} ( - {t}_{ToF} )} = δ ( t - \frac{2 d}{c} ) {e}^{j 2 π {f}_{c} ( - \frac{2 d}{c} )} = δ ( t - \frac{2 d}{c} ) {e}^{- j \frac{4 π d}{λ}} . \qquad ( 5 )$$

In one or more embodiments, δ is Dirac delta function. The resolution using the amplitude is limited by bandwidth as shown by formula (6):

$${d}_{res} = \frac{c}{2 B} . \qquad ( 6 )$$

In various embodiments, dᵣₑₛ is the range resolution, c is the speed of light and B is the bandwidth. However, when analyzing phase data, the resolution is much finer and in the same magnitude as the wavelength (λ). Accordingly, monitoring phase changes over time may provide finer grain analysis such as smaller displacements of the target and/or velocity estimates. This better resolution may allow for gesture recognition, fall detection, or even vital sign monitoring.

In a bi-static radar according to FIG. 2B, the carrier signal (Sₜₓ) produced at the transmitter 202′ has a frequency (fₜₓ) for up-conversion and the carrier signal (Sᵣₓ) received at the receiver 204′ has a frequency (fᵣₓ) for down-conversion. The frequencies (fₜₓ and fᵣₓ) are different. In other words, they may have a slightly different center frequency. The frequency offset (Δf) may be defined by formula 7:

$$Δ f = {f}_{tx} - {f}_{rx} . \qquad ( 7 )$$

Further, the up-conversion and down-conversion signals have uncorrelated initial phases resulting in a phase offset (Δθ) that may be defined by formula 8:

$$Δθ = {θ}_{tx} - {θ}_{rx} . \qquad ( 8 )$$

In one or more embodiments, θₜₓ is the initial phase of the transmitted signal and θᵣₓ is the initial phase of the received signal. Accordingly, the transmitted signal may be represented by formula 9:

$${S}_{tx} ( t ) = I ( t ) {e}^{j 2 π {f}_{tx} t + {θ}_{tx}} . \qquad ( 9 )$$

The received signal may be represented by formula 10:

$${S}_{rx} ( t ) = I ( t - {t}_{TOF} ) {e}^{j 2 π {f}_{tx} ( t - {t}_{TOF} ) + {θ}_{rx}} . \qquad ( 10 )$$

The CIR from the bi-static architecture 220 may be characterized by formula (11):

$${h}_{bi} ( t ) = δ ( t - {t}_{ToF} ) {e}^{j 2 πΔ ft - 2 π {f}_{tx} {t}_{TOF} + Δθ} = {h}_{mono} ( t ) {e}^{j πΔ ft + Δθ} . \qquad ( 11 )$$

Accordingly, the bi-static architecture 220 additionally accounts for the frequency offset (Δf) and the phase offset (Δθ) (e.g., step 908). The frequency offset may result in a linear drift in phase over time. The phase offset or noise may be considered a random number in the characterization.

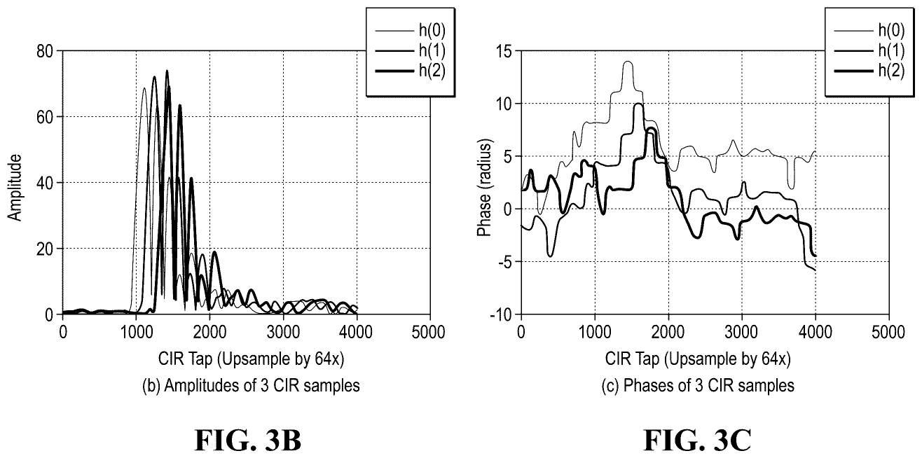

Referring to FIG. 3A, bi-static architecture 300 includes transmitter 302 and receiver 304 that are not physically linked, synchronized, and do not include a shared timing source. For example, a pair of Qorvo DW1000 transceivers may be used as the transmitter 302 and receiver 304. FIGS. 3B-C illustrate three adjacent CIR samples obtained from the bi-static architecture of FIG. 3A with a generally static environment. The CIRs of FIGS. 3B and 3C are not aligned. For example, each sample is offset along the x-axis indicating non-alignment in the time domain. This is associated with the sampling offset. The frequency offset (Δf), described above, is not shown because traditional commodity UWB transceivers may already account for or estimate the frequency offset. For example, this functionality may be useful to perform coherent demodulation.

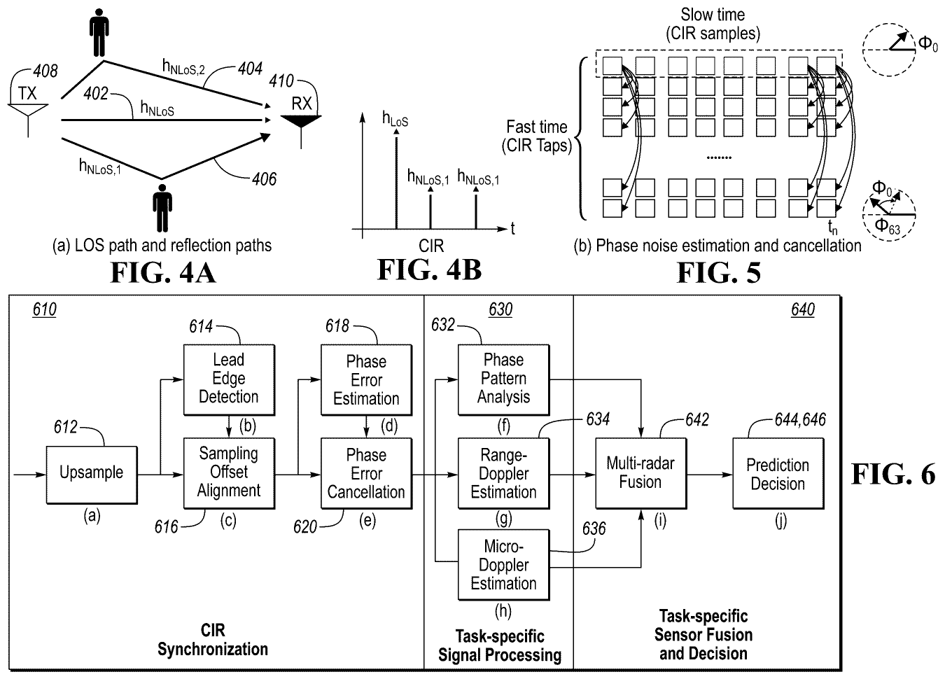

The random phase offset associated with different initial phases is also demonstrated in FIG. 3C as h(0), h(1), h(2) are each randomly offset along the y-axis. The randomness of this offset is particularly challenging. For example, estimation strategies computed in advance may be ineffective. But the phase noise associated with unsynchronized nodes must be identified and distinguished from phase changes associated with the target for finer grain or more particularized processing such as that involved in gesture recognition and vital sign monitoring. This may be achieved by focusing on the first path such as the line-of-sight (LOS) path 402, as shown FIG. 4A. Although described herein with the LOS path as the first path, it should be understood that a non-line-of-sight (NLOS) first path may be used in some circumstances to estimate and cancel phase noise associated with subsequent paths. Generally, CIRs consist of a LOS path 402 and multiple reflected NLOS paths 404 and 406, which may be characterized by formula 12:

$${h}_{bi} ( t ) = {h}_{LOS} ( t ) + {\sum }_{i} {h}_{NLOS , i} ( t ) = {α}_{LOS} δ ( t - \frac{{d}_{LOS}}{c} ) {e}^{- j \frac{2 π {d}_{LOS}}{λ} + Δθ} + {\sum }_{i} {α}_{i} δ ( t - \frac{2 {d}_{i}}{c} ) {e}^{- j \frac{4 π {d}_{i}}{λ} + Δθ}$$

In various embodiments, h_{LOS}(t) is the LOS element and h_{NLOS}(t) represents NLOS elements. By identifying the phase noise associated with the LOS path 402, the phase noise can be estimated and ignored, cancelled, and/or removed from the other NLOS paths. The LOS path 402 travels directly from the transmitter 404 to the receiver 406, as shown in FIG. 4A. Accordingly, the change in phase for the LOS element corresponds to the phase noise (Δθ) as this may, for example, be the only or primary cause. In the same CIR, the NLOS elements suffer from the same phase noise (Δθ) as the LOS element. Thus, the phase of the LOS element is used to estimate the phase error associated with the lack of synchronization or unsynchronized carriers. This estimation may be used to adjust and/or correct the phase of NLOS (reflected) paths.

The LOS path 402 involves the shortest distance (as shown in FIG. 4A) so the LOS path signal is easily separated or extracted from the CIR based on the time domain (e.g., the first or leading tap is the LOS), as illustrated by FIG. 4B. For example, leading-edge detection (LED) techniques such as a LED algorithm may be used to identify the first tap. In a refinement, the phase of the leading edge is obtained and subtracted from all subsequent CIR taps to eliminate the phase noise (Δθ), as shown in FIG. 5. The sampling offset may also be eliminated or normalized by aligning the CIR samples over time based on the leading edge. For example, a synchronized CIR may be characterized by equation (13):

$${h}_{bi - sync} ( t ) = \frac{{\sum }_{i} {h}_{NLOS , i ( t )}}{{h}_{LOS} ( \frac{{d}_{LOS}}{c} )} = \frac{{\sum }_{i} δ ( t - \frac{2 {d}_{i}}{c} ) {e}^{- j \frac{4 π {d}_{i}}{λ} + Δθ}}{{e}^{- j \frac{2 π {d}_{LOS}}{λ} + Δθ}} = {\sum }_{i} δ ( t - \frac{2 {d}_{i}}{c} ) {e}^{- j \frac{4 π {d}_{i}}{λ} + \frac{2 π {d}_{LOS}}{λ}} \qquad ( 13 )$$

As shown above, phase noise (Δθ) is eliminated. In one or more embodiments, d_{LOS} is constant as the distance of the LOS path does not change such that the entire

$$\frac{2 π {d}_{LOS}}{λ}$$

term is merely a constant that does not affect signal processing to estimate relative movements. In other words, only the phase change is significant to such processing.

In various embodiments, synchronization of a sequence or plurality of raw CIR samples is merely a (e.g., first or beginning) step in the process. For example, the radar-like sensing techniques may include one or more of (1) CIR synchronization 610, (2) signal processing 630, and (3) fusion and/or decision-making 640, as shown in FIG. 6.

Again, referring to FIG. 6, each step may have a series of sub-step. For example, synchronization 610 may include upsampling 612, lead edge detection 614 to determine the first path and/or tap index, alignment 616 based on the first path, phase noise estimation 618 based on the phase of the first path, and/or phase error cancellation 620. Signal processing is additional processing beyond synchronization such as task-specific processing to, for example, various data representations. In various embodiments, the signal processing step may include numerous processing techniques (e.g., step 910) such as phase pattern analysis 632, range-doppler estimation 634, and/or micro-doppler estimation 636. In various embodiments, the processing step can be followed by step 640, which includes fusion 642, providing a prediction 644, making a decision, and/or actuating performance 646 (e.g., steps 912 and 914).

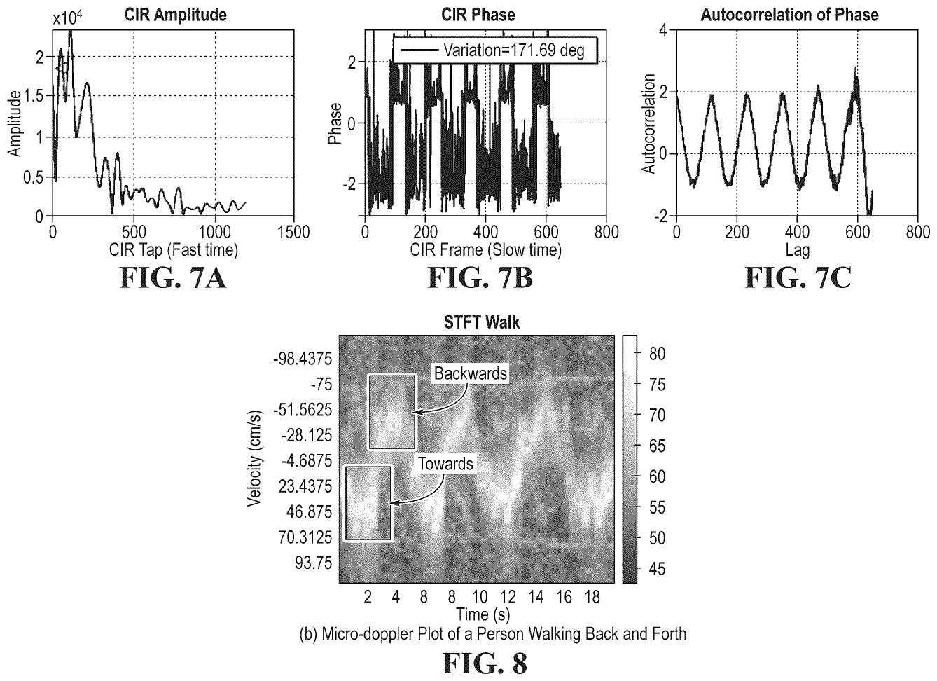

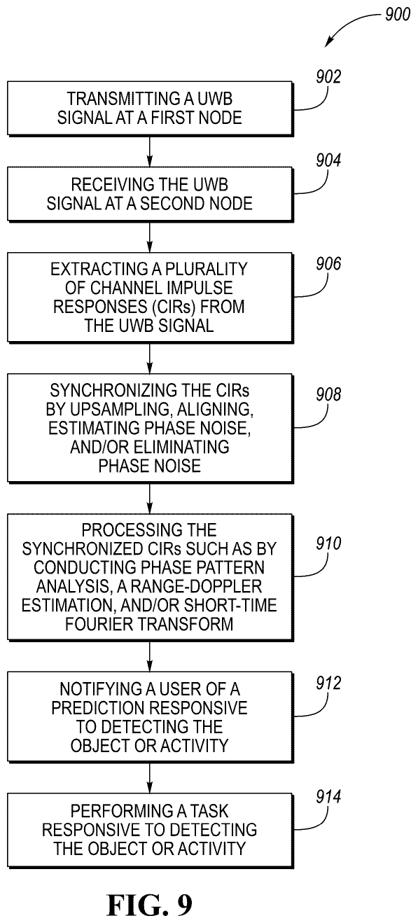

Phase pattern analysis 632 may, for example, be used to sense micro-motions of a target such as breathing, heartbeat monitoring, and/or vibration. In a refinement, micro-motions may refer to movements spanning a distance of no more than 5 cm, or more preferably no more than 1 cm, or even more preferably no more than 0.5 cm. Phase pattern analysis may involve analyzing the frequency domain of the synchronized CIRs. For example, the phase may be transformed into the frequency domain by Fourier transform algorithms such as the fast Fourier transform algorithm (FFT). Once transformed into the frequency domain deviations may be detected and associated with motion. In a refinement, major deviations may signal duress or trigger an alert or alarm. For example, FIGS. 7A-C show phase pattern analysis monitoring breathing using a pair of DW1000 UWB radios. For example, FIG. 7A illustrates a fast time tap amplitude profile which can be used to identify the first path for synchronization and the breathing pattern. FIG. 7B illustrates slow time tap phase profile which shows periodic breathing patterns. FIG. 7C is an autocorrelation of the phase sequence in FIG. 7B for more nuanced breathing pattern recognition. For example, the high correlation in several lags is representative of the periodicity of the phase which may representative of a breathing pattern and/or rate.

Additionally, or alternatively, range doppler estimation may be used such as for localization or tracking of an object. Range doppler estimation may include a short-time Fourier transform (STFT) on each CIR tap to produce a range-doppler plot. Various techniques may be used to identify a target. For example, a constant false alarm rate (CFAR) algorithm may be used to identify a target. The CFAR algorithm may assist in differentiating targets from general noise. This technique may be used to determine the velocity of a target. In one or more embodiments, a Kalman filter may be used for tracking based on this information.

In yet another example, micro-doppler estimations may be used for processing and understanding the environment through the received signal. In one or more embodiments, micro-doppler estimations may be used for analyzing a sequence of motions such as for gesture recognition. Similar to range doppler estimation short-time Fourier transform of the CIRs may be performed to provide a velocity profile with respect to time. FIG. 8 shows velocity profiles from micro-doppler signal processing of a target walking forwards and backwards. For example, at approximately two seconds a forward velocity of approximately 23-70 centimeters per second is detected and at approximately four second a backward velocity of approximately 28-51 centimeters per second is detected.

Multiple possible design architectures are possible. For example, one receiver-multiple transmitters (processing pipeline conducted at one receiver) or multiple receivers-multiple transmitters where each device may take turns transmitting while all others receive (round robin style) may be used. The collection may be transmitted to central device for processing as raw data, after phase cancellation, or after task specific processing. In other words, each node may conduct the processing or a portion thereof individually or the nodes may send the raw data or send partially processed data directly to a central processing unit which may be a single node or a separate computing device. Thus, in yet another embodiment, all devices could perform the entire processing pipeline and inform a central device of a prediction/decision for actuation. In various embodiments machine learning may be employed for additional or further processing such as at the central device.

In one or more embodiments, the functions, steps, and/or algorithms described herein may be performed by a controller (not shown). The controller may include a processor, memory, and/or non-volatile storage. The processor may include one or more devices selected from high-performance computing systems including high-performance cores, microprocessors, micro-controllers, digital signal processors, microcomputers, central processing units, field programmable gate arrays, programmable logic devices, state machines, logic circuits, analog circuits, digital circuits, or any other device that manipulate signals (analog or digital) based on computer-executable instructions residing in the memory. The memory may include a single memory device or a number of memory devices including, but not limited to, random access memory (RAM), volatile memory, non-volatile memory, static random access memory (SRAM), dynamic random access memory (DRAM), flash memory, cache memory, or any other device capable of storing information. The non-volatile storage may include one or more persistent data storage devices such as a hard drive, optical drive, tape drive, non-volatile solid state device, cloud storage or any other device capable of persistently storing information.

The processor may be configured to read into memory and execute computer-executable instructions of the non-volatile storage. Executable instruction may reside in a software module. The software module may include operating systems and applications. The software module may be compiled or interpreted from a computer program created using a variety of programming languages and/or technologies, including, without limitation, and either alone or in combination, Java, C, C++, C #, Objective C, Fortran, Pascal, Java Script, Python, Perl, and PL/SQL.

Upon execution by the processor, the computer-executable instruction of the software module causes the computing platform to implement one or more of the functions, steps, and/or algorithms disclosed herein. Non-volatile storage may also include data supporting the functions, features, calculations, and processes. It should be further understood that numerous other devices may employ certain components as described herein and cooperate with other components.

While exemplary embodiments are described above, it is not intended that these embodiments describe all possible forms encompassed by the claims. The words used in the specification are words of description rather than limitation, and it is understood that various changes can be made without departing from the scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A detection method comprising:
transmitting a UWB signal from a designated transmitter node;
receiving the UWB signal at a separate receiving node;
extracting a plurality of channel impulse responses (CIRs) from the UWB signal;
synchronizing the plurality of CIRs to provide a sequence of synchronized CIRs by:
upsampling each CIR;
aligning the plurality of CIRs;
estimating a phase noise based on a phase of a line-of-sight path signal; and
eliminating the phase noise from each CIR; and
processing the synchronized CIRs to detect an object or activity.

2. The detection method of claim 1, further comprising notifying a user of a prediction responsive to detecting the object or activity.

3. The detection method of claim 1, further comprising performing a task responsive to detecting the object or activity.

4. The detection method of claim 1, wherein processing includes phase pattern analysis of the synchronized CIRs.

5. The detection method of claim 4, wherein processing includes detecting a micro-motion.

6. The detection method of claim 5, wherein the micro-motion is breathing or a heartbeat.

7. The detection method of claim 1, wherein processing includes range-doppler estimation.

8. The detection method of claim 1, wherein processing includes short-time Fourier transform.

9. The detection method of claim 1, wherein processing includes employing a constant false alarm rate algorithm to identify a target.

10. The detection method of claim 1, wherein aligning the plurality of CIRs includes employing a lead edge detection algorithm and aligning the CIRs based on a first path signal.

11. The method of claim 1, wherein estimating the phase noise includes separating the line-of-sight path signal.

12. A method of synchronizing a plurality of ultra-wideband (UWB) nodes for radar-like functionality, the method comprising:
upsampling each channel impulse response (CIR) of a plurality of plurality of CIRs from a UWB signal;
employing a lead edge detection algorithm and aligning the plurality of CIRs based on a lead edge;
determining a phase of a line-of-sight path signal for each CIR; and
eliminating phase error by subtracting the phase of the line-of-sight path signal from each subsequent path signal.

13. The method of claim 12, wherein the line-of-sight path signal is determined using lead edge detection.

14. A distributed ultra-wideband (UWB) radar system comprising:
a transmitting node operable to transmit a UWB signal;
a receiving node operable to:
receive the UWB signal;
extract a plurality of channel impulse responses (CIRs) from the UWB signal;
synchronize the plurality of CIRs to provide a sequence of synchronized CIRs by:
upsampling each CIR;
aligning the plurality of CIRs;
estimating a phase error based on a phase of a line-of-sight path; and
eliminating the phase error;
process the synchronized CIRs to detect an object or movement; and
responsive to detecting an object or movement, actuating an alert corresponding to a prediction or performing a task based on the object or movement.

15. The system of claim 14, wherein the transmitting node is one of a plurality of transmitting nodes.

16. The system of claim 15, wherein the receiving node is one of a plurality of receiving nodes.

17. The system of claim 14, wherein the receiving node is one of a plurality of receiving nodes.

18. The system of claim 17, wherein the plurality of receiving nodes communicates with a central processing unit to extract a plurality of channel impulse responses (CIRs) from the UWB signal, synchronize the plurality of CIRs to provide a sequence of synchronized CIRs, process the synchronized CIRs to detect an object or movement, and/or actuate an alert corresponding to a prediction or perform a task based on the object or movement.

19. The system of claim 17, wherein the plurality of receiving nodes is in communication with a controller operable to employ a data fusion algorithm.

20. The system of claim 14, wherein the receiving node is in communication with a controller operable to extract a plurality of channel impulse responses (CIRs) from the UWB signal, synchronize the plurality of CIRs to provide a sequence of synchronized CIRs, process the synchronized CIRs to detect an object or movement, and actuate an alert corresponding to a prediction or perform a task based on the object or movement.
